(19) United States
(12) Patent Application Publication
(10) Pub. No.: US 2008/0200838 A1
(43) Pub. Date: Aug. 21, 2008

(54) WEARABLE, PROGRAMMABLE AUTOMATED BLOOD TESTING SYSTEM

(76) Inventors: Daniel Goldberger, Boulder, CO (US); Eric Shreve, Louisville, CO (US); Wayne Siebrecht, Golden, CO (US); Benny Pesach, Rosh Haayin (IL); Gidi Pesach, Kfar Vitkin (IL); Gabby Bitton, Jerusalem (IL); Ron Nagar, Tel Aviv (IL); Dalia Argaman, Hod-Hasharon (IL); Stephen Bellomo, Zicron Yacov (IL); Robert Larson, Perkasie, PA (US); Larry Johnson, Pine, CO (US); Jill Klomhaus, Niwot, CO (US)

Correspondence Address: PATENTMETRIX, 14252 CULVER DR. BOX 914, IRVINE, CA, 92604, US

(21) Appl. No.: 11/469,586

(22) Filed: Sep. 1, 2006

Related U.S. Application Data
(63) Continuation-in-part of application No. 11/287,897, filed on Nov. 28, 2005, now abandoned.

(51) Int. Cl. A61B 5/00; A61B 17/32

(52) U.S. Cl. 600/583; 600/300

(57) ABSTRACT

The present invention is a programmable, automated device for measurement and analysis of blood analytes and blood parameters. The device components are preferably combined in a single housing and either programmed to initiate automatic, periodic blood sampling or initiate automatic blood sampling via operator input or in response to a predefined event or in response to a signal from another instrument. The device operates automatically to draw blood samples and analyze the drawn blood samples to obtain the desired blood readings.

### CROSS-REFERENCE TO RELATED APPLICATIONS

The present application is a continuation-in-part of U.S. patent application Ser. No. 11/287,897, entitled “Wearable, Programmable Automated Blood Testing System” and filed on Nov. 28, 2005.

### FIELD OF THE INVENTION

The present invention relates generally to a device and method for monitoring blood parameters and blood constituents, and in particular, to a device and system for portable and programmable periodic measurement of blood glucose and other analytes.

### BACKGROUND OF THE INVENTION

Patient blood chemistry and devices, systems and methods of monitoring patient blood chemistry are important diagnostic tools in patient care. Measuring blood analytes and parameters often yields much needed patient information, allowing for drug administration to be carried out in the proper amounts and time periods. Blood analytes and parameters tend to change frequently, however, especially in the case of a patient under continual treatment, thus making the measurement process tedious, frequent, and difficult to manage.

Diabetes mellitus, for example, can contribute to serious health problems because of the physical complications that can arise from abnormal blood glucose levels. Maintaining a consistent and normal blood glucose level is a challenging and arduous task as the diabetic's blood glucose level is prone to wide fluctuations, especially around mealtime. Many diabetics are insulin dependent and require routine and frequent injections to maintain proper blood glucose levels.

Controlling glucose levels requires continuous or frequent measurements of blood glucose concentration in order to determine the proper amount and frequency of insulin injections. The ability to accurately measure analytes in the blood, particularly glucose, is important in the management of diseases such as diabetes.

Prior art systems have conventionally focused upon manually obtaining blood samples from capillary blood test devices for intermittent use. Such electronic devices are generally handheld and require several manual operations. For example, conventional glucose measurement techniques typically require assembling a clean lancet into a spring-loaded lancing device, triggering the lancing device to puncture a convenient part of the body (normally a fingertip) with a lancet, milking the finger to produce a drop of blood at the impalement site, and depositing the drop of blood on a measurement system (such as an analysis strip to be read via an electronic meter). This lancing method, at typical measurement frequencies of two to four times a day, is both painful and messy for the patient. In addition, the patient must dispose of the blood contaminated material, where proper disposal may be inconvenient.

SureStep® Technology, developed by Lifescan, is one example of a conventional home monitoring system. The SureStep® Technology, in its basic form allows for simple, single button testing, quick results, blood sample confirmation, and test memory. In operation, the SureStep® home monitoring system employs three critical steps to obtain a measurement. In a first step, the blood sample is applied to the test strip. In a second step, the glucose reacts with the reagents in the test strip. The intensity of color formed at the end of the reaction is proportional to the glucose present in the sample. In a third step, the blood glucose concentration is measured with SureStep® meters. Reflectance photometry quantifies the intensity of the colored product generated by the enzymatic reaction. The system is calibrated to yield plasma glucose values.

U.S. Pat. No. 6,192,891, assigned to Beckton, Dickson, and Company, discloses “in a diagnostic and medication delivery system, a unit comprising: a housing, said housing having a first compartment adapted to removably receive and store a medication delivery pen and a second compartment adapted to removably receive and store a lancer; and a monitor integrated in the housing for monitoring a characteristic of a sample of a bodily fluid, wherein said monitor is not integrally attached to said medication delivery pen, such that a user is provided with the flexibility to use different medication delivery pens with said system but only one monitor.”

U.S. Pat. No. 6,849,237, assigned to Polymer Technology Systems, Inc., discloses “a diagnostic apparatus for testing body fluids, comprising: a base having: a slot adapted for receipt of a first test strip; a first display configured to display the concentration of an analyte in a body fluid sample contained in the first test strip; and a docking station adapted to detachably receive a portable tester; and a portable tester detachably mountable to said base, said portable tester having a second display and a port adapted to receive a second test strip containing a body fluid sample, said portable tester operable to test the sample contained in said second test strip when detached from said base.”

The conventional glucose meters described above, however, have substantial disadvantages. Patients often forget, or in some instances forego, conducting and correctly recording their glucose levels as measured by the instrument.

In the light of above described disadvantages, there is a need for programmable, automated systems and methods that can provide comprehensive, accurate, and easy-to-use blood parameter testing. More specifically, what is needed is a programmable, automated system and method for obtaining blood samples at predetermined time intervals or in response to predetermined events for convenient testing of blood parameters and also for data management of measurement results, thus avoiding human recording errors.

What is also needed is a programmable and portable, automated system and method for obtaining blood samples for the convenient testing of blood parameters.

What is also needed is a programmable and wearable, automated system and method for obtaining blood samples for the convenient testing of blood parameters.

### SUMMARY OF THE INVENTION

The present invention is a programmable, automated device for measurement and analysis of blood analytes and blood parameters. The device components are preferably combined in a single housing and either programmed to initiate automatic, periodic blood sampling or initiate automatic blood sampling via operator input or in response to a predefined event or in response to a signal from another instrument, such as an insulin pump signaling an intent to deliver a dose of insulin. The device operates automatically to draw blood samples and analyze the drawn blood samples to obtain the desired blood readings.

In one embodiment, the present invention is an automated blood testing device comprising a sampling and measurement unit for obtaining a blood sample and measuring blood analytes in said sample, wherein the sampling and measurement unit further comprises a plurality of lancet and blood analyte measuring element pairs; and a control unit for controlling the periodic sampling of blood and measurement of blood analytes. Optionally, the control unit is programmable to initiate blood sampling for measurement of blood analytes at pre-determined time intervals or based upon a pre-defined event.

Optionally, the lancet in each pair withdraws blood from a different point for each sample. Optionally, the lancet vibrates to withdraw a blood sample. Optionally, the lancet is a single-use lancet, replaceable, and/or disposable. Optionally, the lancet is contained in a disposable cartridge or cassette. An exemplary blood analyte measurement element is a glucose oxidase test strip. Optionally, the lancet and blood analyte measurement element in each pair is arranged in a “V” configuration. Optionally, each lancet is coated with an anticoagulant or an anesthetic. Optionally, each lancet is provided with a flexible cover that deforms to expose the lancet tip when the lancet is actuated for sampling.

Optionally, the device is wearable. Optionally, the device further comprises an inflatable cuff, which is used for obtaining a blood sample via applying pressure. Optionally, the inflatable cuff comprises a plurality of cavities, wherein each cavity can be individually inflated. Optionally, the inflatable cuff is used for non-invasive measurement of blood pressure. Optionally, the inflatable cuff comprises a warming pad.

In another embodiment, the present invention is directed to an automated device for obtaining a blood sample and measuring blood analytes and blood parameters in said sample, comprising a plurality of lancets in physical proximity for drawing a blood sample; a plurality of blood analyte measuring elements, each in physical proximity to at least one of the lancets; and at least one processor for calculating numerical value of the blood analyte measured by the blood analyte measuring element.

Optionally, the lancet, the blood analyte measuring element and the processor are integrated into a single unit. Optionally, the unit is replaceable and/or disposable. Optionally, the unit is capable of being connected to a physiological parameter monitoring device.

Optionally, the plurality of lancets and said plurality of blood analyte measuring elements are contained in a cassette and wherein the processor is contained a housing capable of detachably receiving the cassette. Optionally, the cassette is replaceable and/or disposable.

Optionally, the cassette is assigned a unique code. Optionally, the unique code can be stored either in mechanical or electrical form. Optionally, the unique code is used by the device to determine if the cassette is authentic, i.e. that the cassette can be used with the device, is authorized to be used with the device, and/or is compatible with the device.

Optionally, the cassette comprises calibration information. Optionally, the calibration information is communicated to the device to enable the at least one processor to accurately calculate the numerical value of the blood analyte measured by said blood analyte measuring element.

The aforementioned and other embodiments of the present invention shall be described in greater depth in the drawings and detailed description provided below.

### BRIEF DESCRIPTION OF THE DRAWINGS

These and other features and advantages of the present invention will be appreciated, as they become better understood by reference to the following Detailed Description when considered in connection with the accompanying drawings, wherein:

FIG. 1 is a block diagram illustrating the major components of an embodiment of the programmable, automated blood parameter testing apparatus of the present invention;

FIG. 2 is a block diagram of one embodiment of a sampling and measurement unit of the programmable, automated blood parameter testing apparatus of the present invention;

FIGS. 3a-3d illustrate a sensor tape as a multiple-layer element, as used in one embodiment of the present invention;

FIG. 4 is an illustration of a sensor cassette as used in the automated blood analysis automated system of the present invention;

Figure is a schematic diagram of an embodiment of a wearable, programmable, automated blood parameter testing apparatus of the present invention;

FIGS. 6a and 6b are schematic diagrams of two embodiments of the automated blood parameter testing apparatus of the present invention, incorporating a cuff;

FIG. 7 illustrates one embodiment of a programmable, automated blood parameter testing apparatus of the present invention;

FIG. 8a, 8b, and 8c depict another embodiment of the automated blood parameter testing apparatus of the present invention, employing lancet and test strip pairs;

FIGS. 8d, 8e, 8f, 8g, and 8h illustrate the operational steps of the automated blood parameter testing apparatus when in use;

FIGS. 9a-9d depict various embodiments of lancet covers that can be used with the automated blood parameter testing apparatus of the present invention;

FIG. 10 illustrates one embodiment of a fluid access interface device that can be used with the automated blood parameter testing apparatus of the present invention; and

FIGS. 11a-11c illustrate another embodiment of a fluid access interface device that can be used with the automated blood parameter testing apparatus of the present invention, wherein a single use transfer tube is integrated with a strip holder and a lancing device.

### DETAILED DESCRIPTION OF THE INVENTION

The present invention is directed towards a programmable, automated device for measurement and analysis of blood analytes and blood parameters. The device components are combined in a single apparatus and either programmed to initiate automatic, periodic blood sampling or initiate automatic blood sampling via operator input or in response to a predefined event or signal from another device. The system operates automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

The present invention is also directed towards a programmable, automated blood parameter testing device that includes a reusable sensor or a plurality of single use sensors that are packaged together in a cassette (hereinafter, referred to as “sensor cassette”) for obtaining blood measurements. The sensors are preferably electrochemical or optochemical sensors, but other options such as sensors that support optical blood measurements (without relying on chemical reactions between the sample of blood and a chemical agent embedded in the sensor) are disclosed. The present invention also discloses apparatuses and methods that employ components of manual test systems (e.g. blood glucose test strips) for use in an automated measurement system.

The present invention is also directed towards programmable, automated devices for measurement and analysis of blood analytes and blood parameters that are wearable. In one embodiment, the present invention is a programmable, automated blood parameter testing device that is advantageously integrated with a conventional pressure cuff or bladder. The inflatable bladder may optionally be employed for squeezing blood from the measurement site and also enables measurement of blood pressure non-invasively, in addition to the capillary blood parameter.

The present invention is also directed towards an integrated, automated blood parameter measurement and analysis system that employs a method of data transmission between the automated measuring system and portable monitors.

In addition, the present invention is directed towards features of the automated blood analysis and measurement system, such as, but not limited to storage of measurement results for trending or later download and alerts or alarms based on predefined levels or ranges for blood parameters.

As referred to herein, the terms “blood analyte(s)” and “blood parameter(s)” refers to such measurements as, but not limited to, glucose level; ketone level; hemoglobin level; hematocrit level; lactate level; electrolyte level (Na⁺, K⁺, Cl⁻, Mg²⁺, Ca²⁺); blood gases (pO₂, pCO₂, pH); blood pressure; cholesterol; bilirubin level; and various other parameters that can be measured from blood or plasma samples.

In one embodiment, the integrated, automated blood parameter analysis and measurement system comprises an automated blood parameter testing apparatus for measuring blood glucose levels.

Reference will now be made in detail to specific embodiments of the invention. While the invention will be described in conjunction with specific embodiments, it is not intended to limit the invention to one embodiment. Thus, the present invention is not intended to be limited to the embodiments described, but is to be accorded the broadest scope consistent with the disclosure set forth herein.

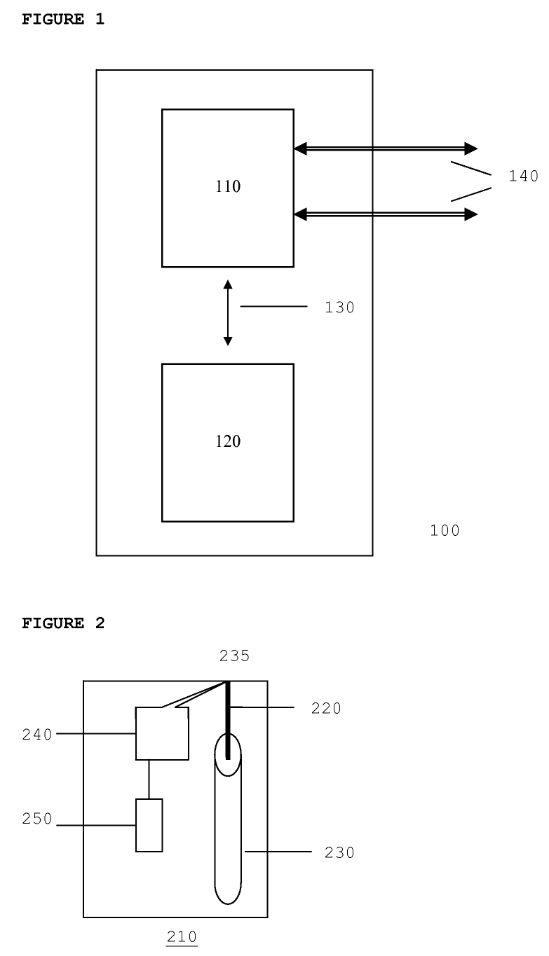

FIG. 1 is a block diagram illustrating the major components of an embodiment of the programmable, automated blood parameter testing apparatus of the present invention. Referring to FIG. 1, automated blood testing device 100 comprises a programmable control unit 110 for controlling the automatic operation of the system and a sampling and measurement unit 120 for obtaining the blood sample and measuring the analytes. The programmable control unit 110 enables automated blood sampling and analysis at predetermined intervals or time periods or in response to an event or operator input or signal from another device. In addition, the programmable control unit 110 can optionally be programmed to initiate blood sampling and measurement based upon a 24-hour time clock. Thus, the patient's blood sampling can be scheduled to record measurements throughout the day, at the same time each day, or can be changed according to an individual daily schedule. For example, a measurement may be scheduled for predetermined time periods which include, but are not limited to, one-, two-, and four-hour time periods.

For example, but not limited to such example, an operator or patient can program the unit to automatically measure blood analytes via initiation of a blood sampling and measurement unit 120 every four hours. It is also possible to program measurements at longer or shorter predetermined intervals or in response to an event or a signal from another device. In addition, the operator or patient can initiate on demand testing. Programmable control unit 110 enables the display of test results as soon as the blood sample reaches the measuring element.

In one embodiment, control unit 110 comprises a general purpose programmable microprocessor unit (not shown), as are well known to persons of ordinary skill in the art. In an alternate embodiment, control unit 110 comprises a state machine implemented in software and at least one processor. The programmable control unit 110 communicates with sampling and measurement unit 120 via an internal communication link 130. Internal communication link 130 may either be wired or wireless and may be based on a digital data link or on analog signals. Besides controlling and synchronizing functions for proper automated operation of the automated blood testing device 100, control unit 110 also includes required alert and built-in test capabilities. For example, but not limited to such example, the programmable control unit includes alert features to detect cuff inflation and lancet position for accurately obtaining a blood sample. Programmable control unit 110 also enables the user to define a reference range or reference values for the blood parameters measured by automated blood testing device 100. Thus, if a measurement is above or below the defined range or values, control unit 110 issues an alarm.

Programmable control unit 110 is also preferably equipped with external communication links 140 that may optionally include interfaces to external automated systems such as, but not limited to, portable monitors, printers, hospital data network(s), external processors and display units, and other monitoring automated systems. The connection between the control unit and the various possible external units can be made via any of the known wired or wireless communication methods, as are well-known in the art.

FIG. 2 is a block diagram of one embodiment of a sampling and measurement unit of the programmable, automated blood parameter testing device of the present invention. In one embodiment, blood sampling and blood analyte measurement means is embodied in a disposable cartridge 210. Disposable cartridge 210 preferably comprises a lancet 220, for piercing skin to obtain a blood sample. Lancet 220 is housed in an automated launching mechanism 230 that launches the lancet 220 when an indication is made that a blood sample needs to be obtained, allows the lancet 220 to pierce the skin, and retracts the lancet 220 after the blood sample is obtained. The automated launching mechanism 230 may be mechanical (such as spring or cam driven) or electrical (such as electromagnetically or electronically driven). In a preferred embodiment, automated launching mechanism 230 is a spring-loaded launching mechanism. Lancet 220 is completely shielded within the launching mechanism 230 when it is not in position for lancing.

Disposable cartridge 210 may contain a single lancet 220 for single patient use or optionally, a plurality of lancets, wherein the lancet is replaced for each measurement. Further, the system may be programmed to pierce the same spot on the skin for every measurement or to target a different spot with each measurement. In an exemplary embodiment, adjacent spots are 1 mm or more apart.

At the point where the lancet pierces the skin, disposable cassette 210 also contains a narrow opening 235 leading to reservoir 240. Narrow opening 235 enables capillary forces to channel the blood sample into reservoir 240. From reservoir 240, the blood sample is carried through at least one small passage, to the blood analyte measuring element 250 contained within cartridge 210. In an alternative embodiment, blood analyte measuring element 250 may be integrated with lancet 220. Further, in another alternative embodiment, the narrow opening in fluid communication with blood as it is sampled may be integrated into the blood analyte measuring element 250.

Referring back to FIG. 2, in one preferred embodiment blood analyte measuring element 250 is a glucose oxidase test strip, preferably disposable, as are well-known to those of ordinary skill in the art. In another embodiment, blood analyte measuring element 250 is a sensor for performing blood analyte measurements. A single pre-calibrated and reusable sensor may be employed. In another embodiment, a plurality of single use sensors may be employed. Each single-use sensor is advanced sequentially and positioned for direct contact with a blood sample through an advancement means.

In one embodiment, the sensor is an electrochemical sensor capable of detecting the presence of and enabling the measurement of the level of an analyte in a blood sample via electrochemical oxidation and reduction reactions at the sensor. In another embodiment, the sensor is an optochemical sensor capable of detecting the presence of and enabling the measurement of the level of an analyte in a blood or plasma sample via optochemical oxidation and reduction reactions at the sensor.

In another embodiment the sensor may optionally include a surface or miniature container, such as but not limited to a capillary tube, enabling storage of the blood sample for optical measurements. In this embodiment, both a light source and a light detector are used for measuring the blood analyte based on reflected, transmitted or other known optical effects such as Raman Spectroscopy, NIR or IR Spectroscopy, FTIR, fluoroscopy, or RF impedance.

When multiple single-use sensors are used, one of the various methods available for packaging multiple sensors may be employed. Packaging options preferably include, but are not limited to: embedding a plurality of sensors in a multi-layered tape structure encapsulated in a compact cassette formation; attaching a plurality of sensors to a tape; or packaging a plurality of sensors in a drum that enables singular selection of a sensor.

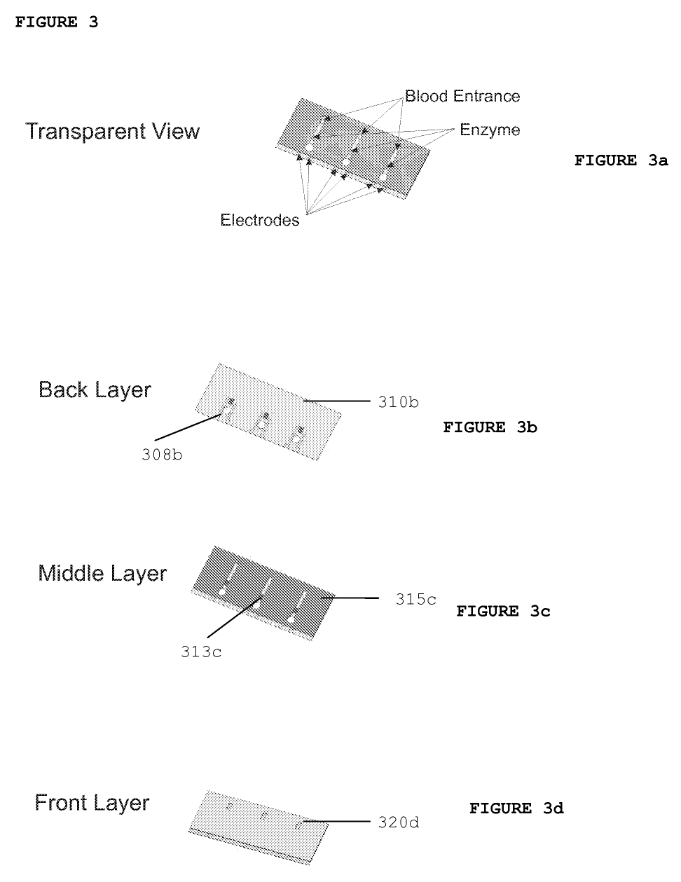

FIGS. 3a-3d illustrate a sensor tape as a multiple-layer element, as used in one embodiment of the present invention. FIG. 3a illustrates a transparent view of the multi-layer sensor tape as used in one embodiment of the present invention, and described in further detail below. FIG. 3b depicts the back layer of the sensor tape; FIG. 3c illustrates the middle layer of the sensor tape; and FIG. 3d illustrates the front layer of the sensor tape as used in one embodiment of the present invention. The sensor tape preferably comprises at least one sensor, and even more preferably comprises a plurality of sensors.

In one arrangement, the sensor tape comprises a front layer 320d (shown in FIG. 3d), a middle layer 315c (shown in FIG. 3c), substantially coplanar with the front layer, that is capable of transporting a blood sample by means of at least one capillary channel 313c and further includes a suitable enzyme coating; and a back layer 310b (shown in FIG. 3b), underlying the middle transporting layer, that comprises a plurality of electrochemical sensor electrodes 308b for sensing required blood analytes such as, but not limited to glucose. Positioned at one end of the at least one capillary channel in the middle transport layer is a hole provided for an air outlet.

The front layer 320d of the sensor tape, and thus each sensor, may optionally be coated with a membrane for blocking the enzyme layer. When using a membrane coating to block the enzyme layer, the sensor measures the plasma analyte level, such as plasma glucose level instead of the blood analyte level.

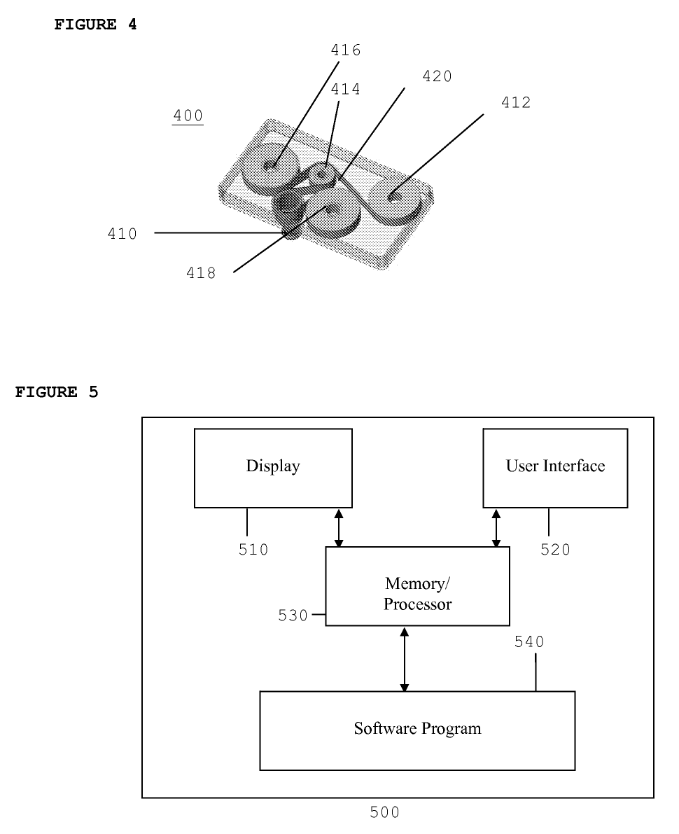

FIG. 4 is an illustration of a sensor cassette as used in the automated blood analysis automated system of the present invention. Single use sensors are preferably packaged into a sensor cassette that is replaced periodically. One such cassette 400 is shown in FIG. 4. In one embodiment, the sensor cassette 400 is assembled as a part of the cartridge containing lancets and the entire assembly is disposable. In another embodiment, the cassette 400 is sterile or provided in a sterile package.

The sensor cassette 400 consists of an advancement mechanism comprising at least one cylindrical element 410 that rotates the sensor tape 420 to bring a sensor in contact with the blood sample. In one embodiment, a plurality of cylindrical elements are used to hold, and permit the movement of, spools of sensor tape 420, including a first cylindrical element 412 to hold a spool of unused sensor tape, a second cylindrical element 414 to permit the movement of unused sensor tape to a third cylindrical element 410 that places the unused sensor tape in fluid communication with a blood sample, a fourth cylindrical element 418 to receive used sensor tape, and a fifth cylindrical element 416 to hold additional sensor tape.

Thus, between measurements, the plurality of cylindrical elements under programmatic control by a central processor, moves the sensor tape forward, thereby replacing a used sensor in the previous measurement with a new sensor. In one design, the sensor cassette also stores the consumed test supplies and sample waste. An external waste container (not shown) may optionally be used to store the waste fluid and/or consumed test supplies.

In addition, the sensor cassette may optionally include different types of single use sensors in one cassette, wherein each sensor is capable of measuring a different type of blood analytes or blood parameters. In this case, sensor selection is made based upon either operator programming or selection before usage. In another optional embodiment, the sensor cassette may include a plurality of cassettes, each comprising a different type of sensor. The same automated blood sampling means is used for each measurement. In another embodiment, each sensor cassette can be pre-calibrated prior to use, i.e. at the point of manufacture.

In another embodiment, the disposable elements are mechanically, electrically, or otherwise keyed to mate with the reusable elements. Mechanical keys can take the form of a variety of three-dimensional, mating shapes, including, but not limited to cylinders, squares, or polygons of various configurations. Electrical keys can be of either analog or digital encoding schemes. Coding information may be transmitted by conventional electrical interfaces (connectors) or via short distance radiofrequency (RF) methods. Software keys may be in the form of a bar code or other passive encoding means. Coding information may be transmitted electrically, optically or by various means known to those skilled in the art.

FIG. 5 illustrates one use of a monitor in conjunction with the programmable, automated blood parameter testing device of the present invention. In one embodiment, the automated blood testing device is connected, either via wired links or wireless links, to a portable, optionally hand-held, monitor. Referring to FIG. 5, monitor 500 may comprise a computing automated system such as, but not limited to, a personal digital assistant (PDA), electronic notebook, pager, watch, cellular telephone and electronic organizer. Signals representing blood parameter data obtained from the patient are presented to the monitor which includes both a conventional processor and memory core 530, a display 510 and human interface means 520, including a mouse, touch screen (responsive to human touch or a special pen-like device), keyboard, or any other form of inputting data. Using interface means 520 a user may program the device for automatic testing of blood at specified time intervals. The monitor is also provided with a memory 530 to facilitate data archiving and retrieval as may be required.

Optionally, various parameter data from the automated blood testing system may be correlated and analyzed in order to indicate the overall patient condition and/or to indicate critical conditions that require attention. In one embodiment, the control unit of the automatic blood parameter testing device performs this data analysis and/or data correlation. In another embodiment, monitor 500 is equipped with software program 540 for data analysis and correlation. Additionally, software program 540 also supports calculation of trends using look-up tables and algorithms based on measurement history. The results of data analysis and interpretation performed upon the stored patient data by the monitor may optionally be displayed in the form of a paper report generated through a printer (not shown) associated with the monitor 500, besides being displayed on the monitor screen 510.

Software 540 uses a blend of symbolic and numerical methods to analyze the data, detect clinical implications contained in the data and present the pertinent information in the form of a graphics-based data interpretation report. The symbolic methods used by the software encode the logical methodology used by doctors as they examine patient logs for clinically significant findings, while the numeric or statistical methods test the patient data for evidence to support a hypothesis posited by the symbolic methods which may be of assistance to a reviewing physician.

Optionally, the processed data may be transmitted from the monitor to a central monitoring station when the automatic blood parameter testing device is used in a hospital environment. The central monitoring station maintains a record of all physiological parameters measured over a period of time from different patients. Thus, a plurality of monitors can communicate with the central monitoring station to supply data from various automated blood parameter testing apparatuses.

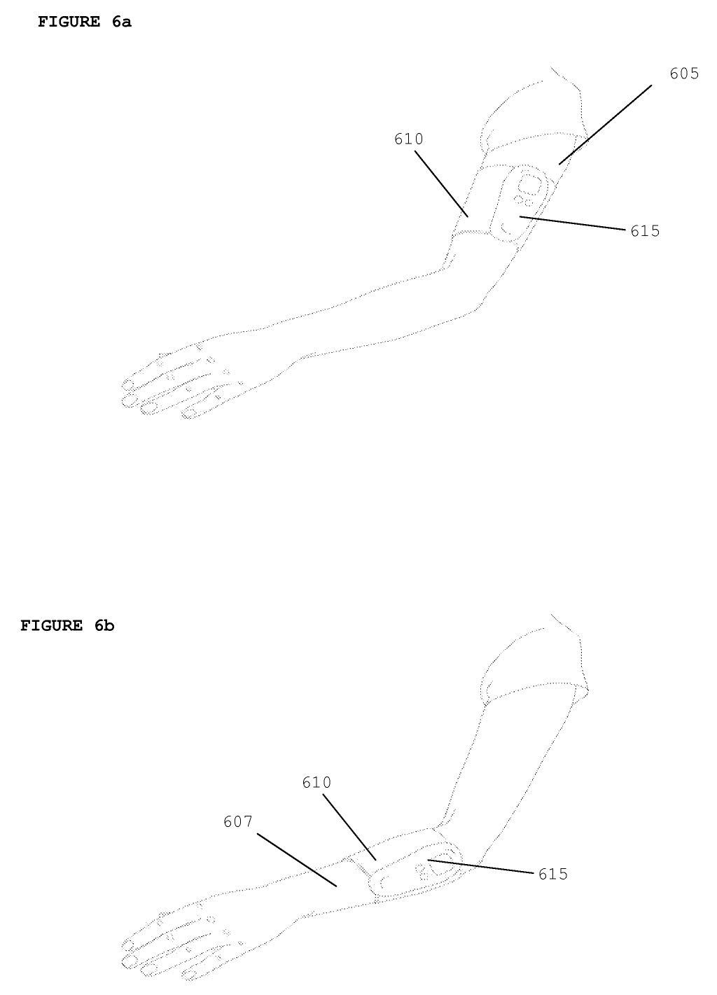

FIGS. 6a and 6b are schematic diagrams of two embodiments of the wearable, programmable, automated blood parameter testing apparatus of the present invention. As shown in FIGS. 6a and 6b, in the wearable embodiments of the device of the present invention, the automated blood testing device 615 is physically attached to a wearable cuff 610. The wearable cuff 610 may be placed on any suitable location of the body, as in, but not limited to, the patient's forearm 607 or patient's upper arm 605. The wearable cuff is preferably secured with an arm band or other suitable attachment mechanism. Other sites, for example the finger, abdomen and leg, are also appropriate for measurement.

In one embodiment the wearable cuff 610 is an inflatable cuff or bladder such as that used with conventional noninvasive blood pressure measuring automated systems.

In one embodiment, the inflatable cuff mechanism is employed for non-invasive measurement of blood pressure. The inflatable cuff acts to occlude blood flow in the underlying artery. This technique of blood pressure measurement is well known in the art, as will not be described in detail herein.

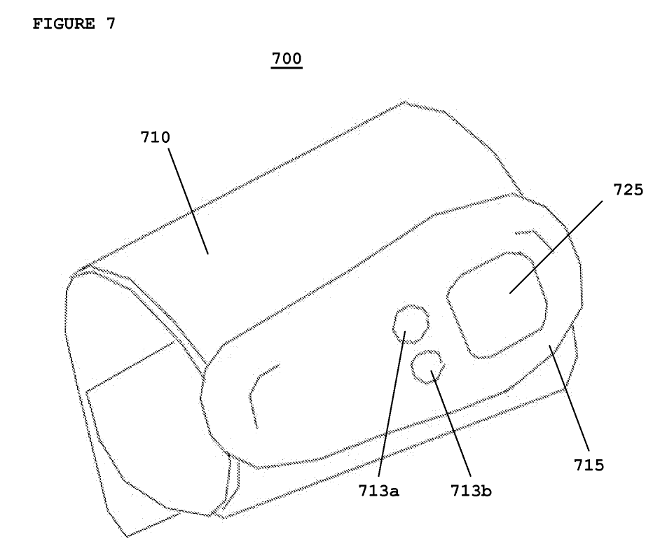

FIG. 7 is a diagram of one embodiment of the automated blood parameter testing apparatus 700 of the present invention, incorporating the testing device 715 and a pressure cuff 710. The device 715 is operated using control buttons 710a and 710b. Display screen 725 is used to monitor the operation of the device 715.

Now referring back to FIG. 2, and also referring to FIG. 7, the operational steps of an integrated pressure cuff and programmable blood testing device are described. When start button (such as 710a) is depressed, the pressure cuff 710 begins to inflate. Substantially simultaneously, automated launching mechanism 230 is actuated, advancing lancet 220, causing lancet 220 to pierce the skin, and retracting lancet 220 after piercing the skin. The inflated pressure cuff facilitates squeezing the blood from the wound in the skin. The blood sample is then collected in reservoir 240, where it was transported via a narrow channel to blood analyte measuring element 250.

In another embodiment of the automated blood parameter testing apparatus of the present invention, a plurality of lancet and test strip pairs is employed, optimally positioned relative to one another, to facilitate effective skin access, blood sampling, sample delivery to the measurement element, and ease of measurement. In addition, an inflatable arm-band or cuff is employed to both facilitate delivery of an optimal blood sample to the device and provide a blood pressure measurement.

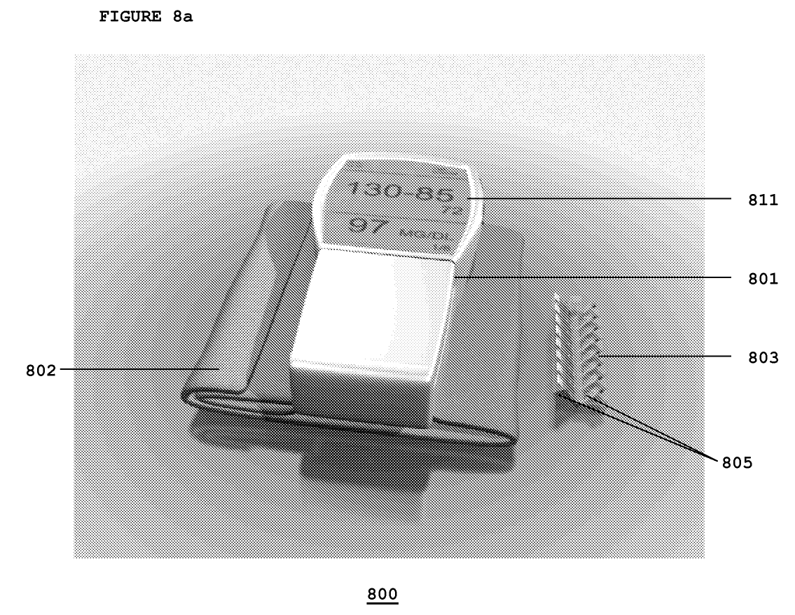

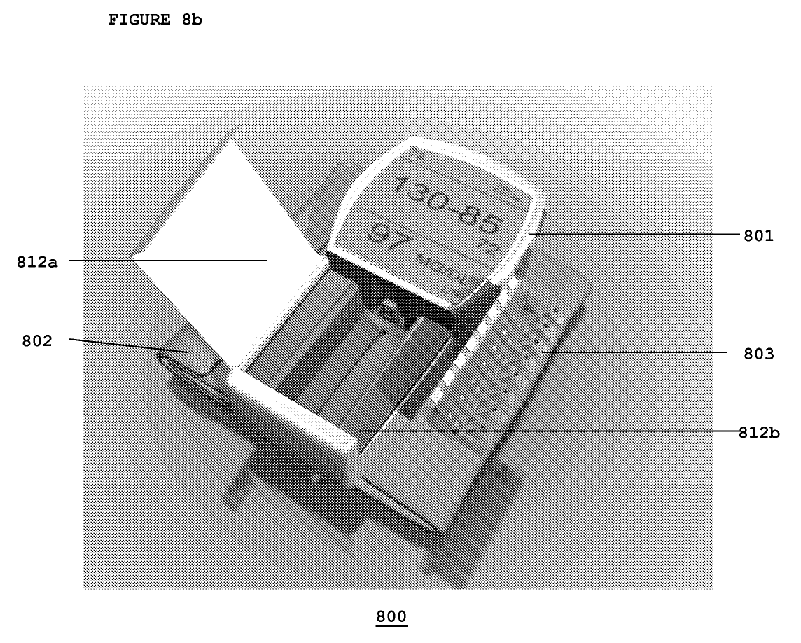

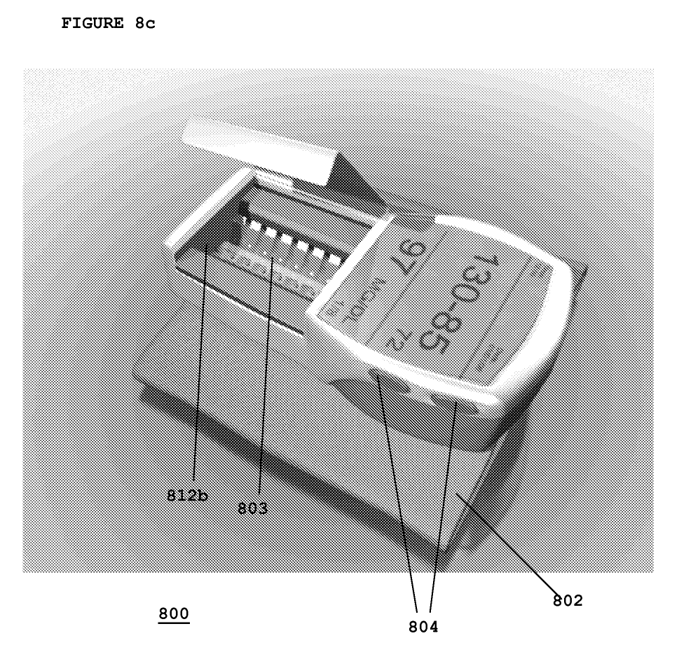

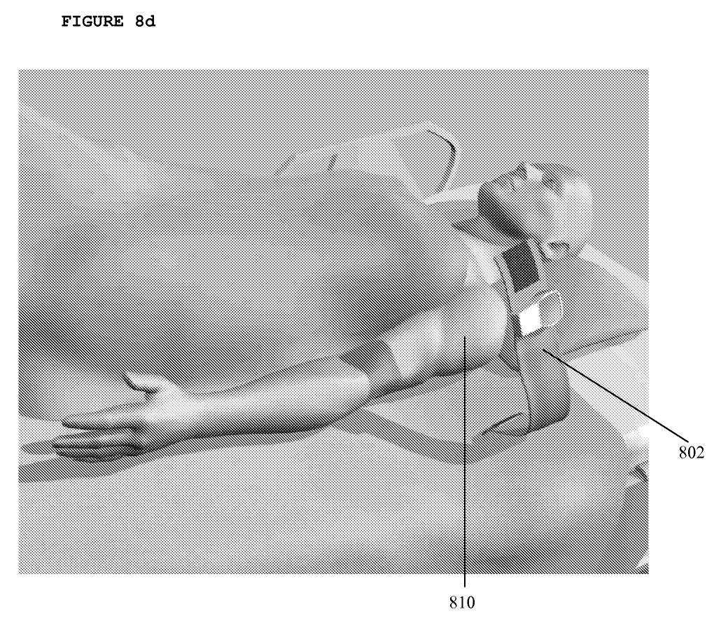

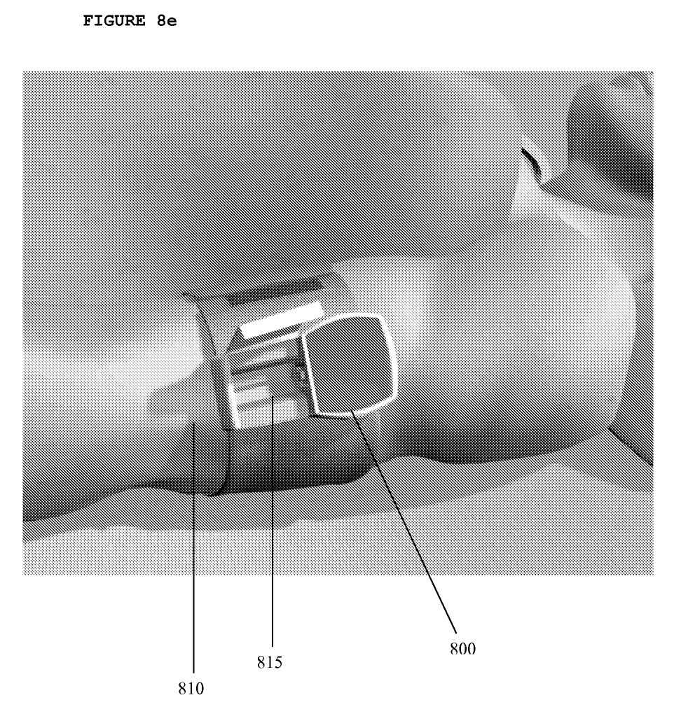

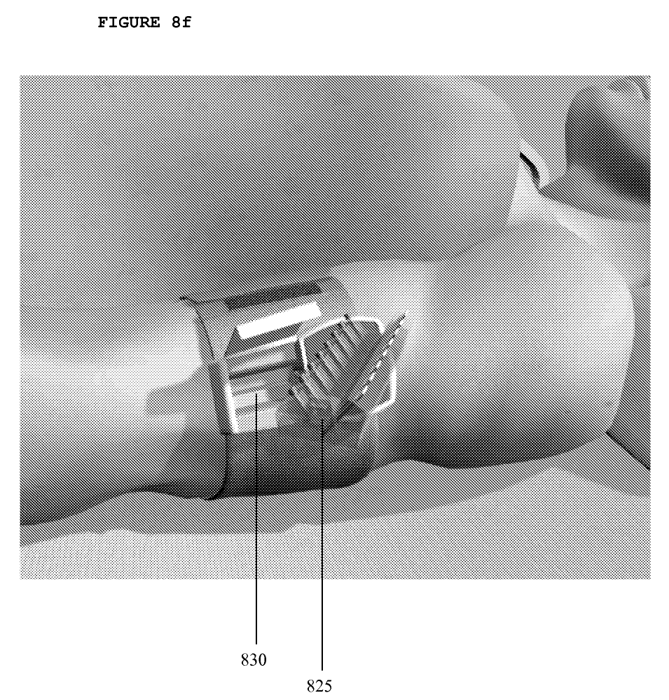

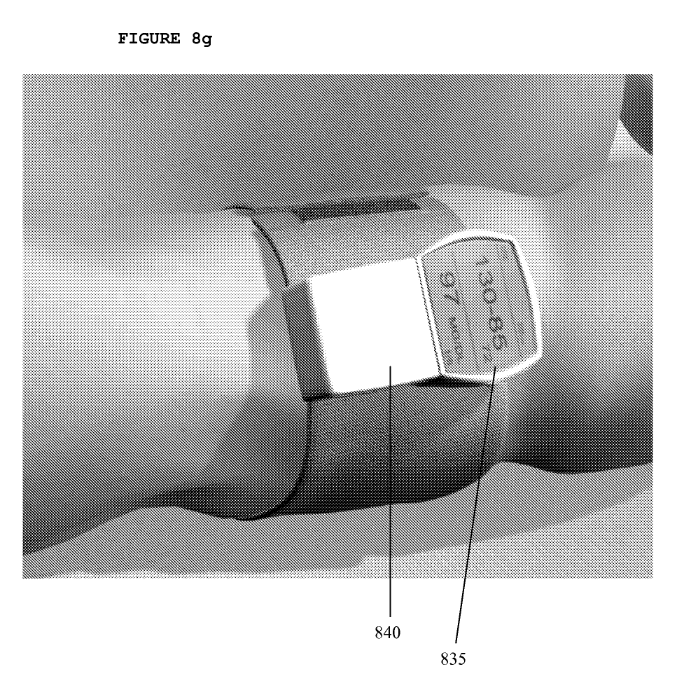

Referring now to FIG. 8a, one embodiment of the wearable, programmable, automated blood parameter testing apparatus 800 of the present invention is illustrated. Automated blood parameter testing apparatus 800 is capable of measuring blood pressure and at least one blood analyte. In one embodiment, apparatus 800 is employed to measure blood glucose levels. In another embodiment, apparatus 800 is employed to measure both blood glucose and blood pressure. One of ordinary skill in the art would appreciate that the apparatus may be modified to allow for measurement of other blood analytes in conjunction with the blood pressure measurement.

As shown in FIG. 8a, apparatus 800 comprises portable housing unit 801 and inflatable arm-band or cuff 802. The blood pressure cuff can be inflated and deflated up to any pressure typically used in the art, such as, but not limited to, a pressure of 240 mm Hg. In one embodiment, the inflatable arm-band or cuff 802 is used to obtain a blood pressure measurement, as shown on display screen 811. In another embodiment, the apparatus 800 is used to obtain a patient's pulse rate.

In another embodiment, the inflatable arm-band or cuff 802 is used to facilitate access to a reliable and consistent blood sample by applying pressure to the sample site. In addition, different blood pressure and blood sampling methods may be employed in order to obtain a reliable and consistent sample.

Thus, in one embodiment, the blood testing apparatus of the present invention employs the inflatable arm-band or blood pressure cuff to optimize blood sampling. In addition, the use of pressure helps alleviate patient discomfort during sampling.

For example, the pressure cuff and the lancet in the apparatus may be operated in, but is not limited to, any of the following sequences: 1) puncture first, then inflate; 2) inflate first, then puncture; 3) inflate, wait for a pre-determined time period, then puncture; 4) inflate, wait for a predetermined time period, puncture, deflate, inflate again; 5) inflate to 80 mm Hg, puncture, inflate to 150 mm Hg, 175 mm Hg, then 200 mm Hg.

In another embodiment, the cuff is double pressurized at the time of blood sampling, to facilitate withdrawal of a blood sample quickly. In yet another embodiment, the blood pressure cuff is sectioned into multiple cavities or channels, so that each channel may be individually modulated or pumped to control blood flow. This mechanism yields a physiological result similar to the act of massaging an area of the arm to stimulate blood flow and assist in the withdrawal of a blood sample. Optionally, the pressure cuff may include a warming pad to improve blood flow and increase the amount of arterial contribution.

Optionally, the blood testing apparatus of the present invention may include a foam barrier between the device and skin. As the pressure cuff is inflated, the foam barrier compresses and seals. Thus, when the cuff pressure is released, the foam expands and absorbs any additional/residual blood not used in sampling or testing. The use of a foam barrier also makes the apparatus more comfortable to wear.

Portable housing unit 801 includes a memory (not shown) for storing historical measurements of any physiological parameter. Such measurements may include prior glucose measurements, prior blood pressure measurements, prior pulse rate measurements, the timing of measurements made, the frequency of measurements made, the relative change of glucose measurements over time, the relative change of blood pressure measurements over time, the relative change of pulse rate over time, or any mathematical relationship therebetween. Each of said measurements can be stored individually or in relation to each other in a table format or other relational data structure.

Portable housing unit 801 further includes a display 811 for displaying measured readings, such as, but not limited to, blood pressure and blood glucose. In addition, display 811 may display the pulse rate. Optionally, display 811 the date and time of the measurement, which is recorded in the memory of the device 800.

The automated blood parameter testing apparatus of the present invention further comprises a disposable cartridge 803, employed to house the blood sampling and parameter measuring elements. In one embodiment, cartridge 803 comprises at least one lancet and test strip pair 805 for blood sampling and analyte measurement. Cartridge 803 may contain any number of lancet and test strip combinations, provided the resulting cartridge structure is still physically compatible with portable housing unit 801. The lancet and test strip pairs 805 are advantageously positioned to facilitate effective skin access, blood sampling, sample delivery to the measurement element, and ease of measurement. In one embodiment, cartridge 803 is disposable. Cartridge 803 is described in greater below with respect to the operational characteristics of the automated blood sampling device of the present invention.

The lancet can be any sharp protrusion capable of piercing skin, such as a needle or any variation thereof. The lancet comprises a projecting body, preferably made of stainless steel, capped with a thermoplastic portion that serves as a means to hold and manipulate the lancet. However, one of ordinary skill in the art would appreciate that other materials can be used.

In one embodiment, each lancet is fitted with a plastic cover that ensures the sterility of the sharp, piercing tip of the lancet. Optionally, the lancet cover may also be used to cover the piercing tip after the lancet is used to eliminate secondary skin pricks. In one embodiment, the lancet cover is spring-loaded and facilitates lancet actuation by acting as a return spring. In such an embodiment, the lancet cover is movably attached to the lancet. In another embodiment, the lancet cover is an elastomeric cover that is pushed out of the way by the act of moving the sharp piercing tip of the lancet toward the patient's skin. In yet another embodiment, the lancet cover is a mechanically actuated by the pressure cuff, thereby moving out of the way of the piercing tip at an appropriate measurement time.

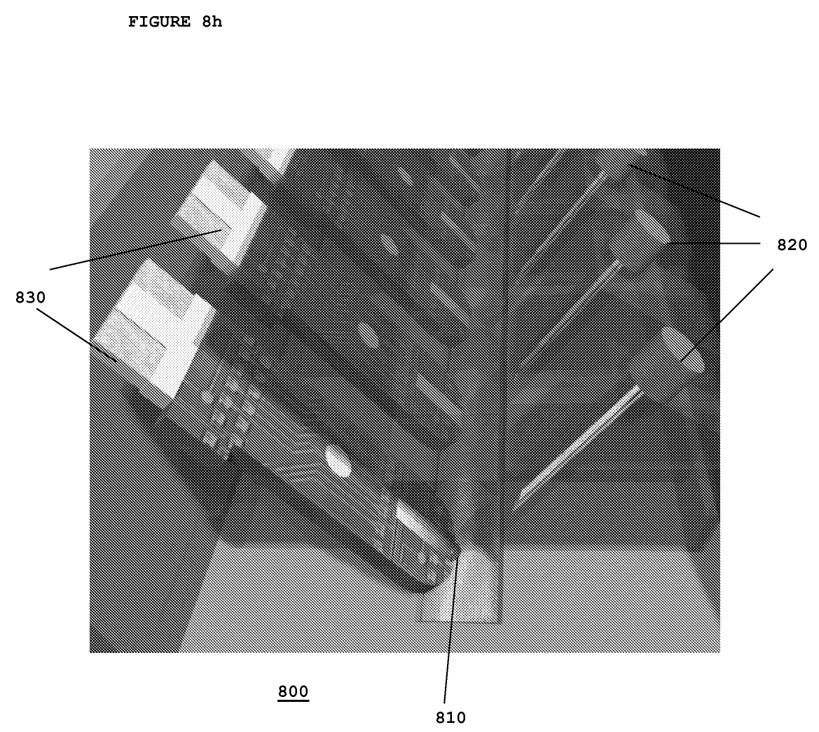

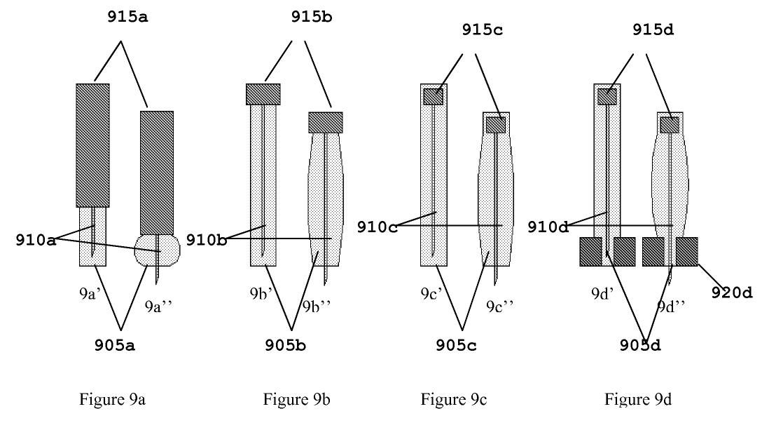

FIGS. 9a-9d depict various embodiments of lancet covers 905a-d that can be used with the automated blood parameter testing apparatus of the present invention. In one embodiment, the lancet cover 905a-d comprises a flexible, pliable material such as, but not limited to, isoprene or silicone, which allows the cover to bend and/or deform to expose the sharp tip of the lancet 910a-d when the lancet 910a-d is actuated. After actuation and withdrawing of a blood sample, the lancet covers 905a-d returns to the original shape and position to seal and cover the used lancet tip. Optionally, a stabilizing base 915a-d and/or tip guide 920d can be incorporated into the device.

FIGS. 9a′, 9b′, 9c′, and 9d′ illustrate the positions of the lancet covers when they are sealed within the lancet tip. FIGS. 9a″, 9b″ 9c″, and 9d″ illustrate the positions of the lancet covers when the lancet is actuated and the lancet cover is deformed. The lancet cover may either be applied as finished material or may be over-molded to the lancet. FIGS. 9c and 9d depict lancet cover designs wherein the cover is a complete over-mold of pliable material. More specifically, FIG. 9d illustrates one embodiment where the lancet cover can act as both a lancet cover and return “spring”, as described above. Therefore, the lancet housing can be molded such that, when actuated, the lancet pushes forth through the housing and then, when pressure is taken away, the lancet housing itself causes the lancet to move back into the housing. Generally, however, the present invention is directed toward any method or structure for individually actuating a lancet, including any spring loaded, electromechanical, or solenoid mechanism that permits the lancet to be “launched” toward the patient's skin upon any signal.

Lancets can be placed into an appropriate piercing position through a number of methods. In one embodiment, the lancets are pre-assembled to be positioned in the appropriate place when installed in the meter housing, provided the housing is positioned appropriately on the patient's arm. In another embodiment, the lancets are positioned in the appropriate piercing position by a positioning mechanism that indexes a lancet location to a preferred measurement site. In one exemplary embodiment, the positioning mechanism may operate by optically aligning the piercing position with a pre-determined preferred measurement location.

The testing strip can be any form of optical or electrical sensing device capable of accepting blood and emitting a signal or a color change indicative of the analyte level within the blood. In one embodiment, the testing strip is a single use electrochemical sensor capable of detecting the presence and/or measuring the level of an analyte in a blood sample via electrochemical oxidation and reduction reactions at the sensor. The electrochemical sensor provides electrical input signal(s) to a signal analyzer, which converts these signal(s) to a correlated usable output, which can be, but is not limited to, an amount, concentration, or level of an analyte, such as glucose, in the patient blood sample. A control unit ensures that electrochemical sensor is maintained in direct contact with the blood sample until the electrical input signals reach a steady state condition, and the signal analyzer measures the required blood analyte(s) and blood parameter(s). The required time period for sensor to be in contact with a blood sample in order to enable the measurement is on the order of seconds.

In another embodiment the electrochemical sensor comprises both a working and a counter enzyme electrode. A counter electrode refers to an electrode paired with the working enzyme electrode. A current equal in magnitude and opposite in sign to the current passing through the working electrode passes through the counter electrode. As used in the present invention, the counter electrode also includes those electrodes which function as reference electrodes (i.e., a counter electrode and a reference electrode may refer to the same electrode and are used interchangeably).

Electrochemical sensors are provided in suitable form for obtaining the desired blood chemistry measurements. In one preferred embodiment of the present invention, the blood glucose level is measured. Electrochemical sensors that can be used for measuring blood glucose level preferably comprise the same type (but not limited to such type) as the sensors currently used in finger sticks for glucose measurement. In this case, a single use sensor provides electrical potentials having a magnitude representing concentration of glucose in the blood.

Another embodiment of a sensor used with the automated blood analysis device of the present invention is a single use optochemical sensor capable of detecting the presence and/or enabling measurement of the level of an analyte in a blood/plasma sample via optochemical oxidation and reduction reactions at the sensor. For example, when using enzymatic reactions to measure a blood analyte, a component is added to the enzymes, which results in an optically measurable color change as a product of the reaction. Either an optical detector or a combination of a light source and an optical detector are used for measuring the blood analyte by measuring the color, and more particularly, color change, at the sensor.

In another embodiment the sensor may optionally include a surface or miniature container, such as but not limited to a capillary tube, acting as a cuvette for optical measurements. In this embodiment, both a light source and a light detector are used for measuring the blood analyte based on reflected, transmitted or other known optical effects such as Raman Spectroscopy, NIR or IR Spectroscopy, FTIR, fluoroscopy, or RF impedance or the like. It should be appreciated that the terms sensing element, blood analyte measuring element, and testing strip are used interchangeably herein.

Within the cartridge, the lancet is positioned relative to the testing strip to allow for a) the unimpeded movement of the lancet back and forth from the patient's skin and b) clear access by the testing strip to the resulting blood droplet, generated by the action of the lancet. In one embodiment, the lancet and test strip pairs are optimally positioned relative to one another, to facilitate effective skin access, blood sampling, sample delivery to the measurement element, and ease of measurement. For example, as shown in 8h, the lancet 820 can be in a V-configuration relative to the testing strip 830. This configuration enables the formation of a channel on the surface of the skin 810, such that when a lancet pricks the skin 810 to draw blood, the blood sample is automatically transported towards the test strip 830 via capillary forces.

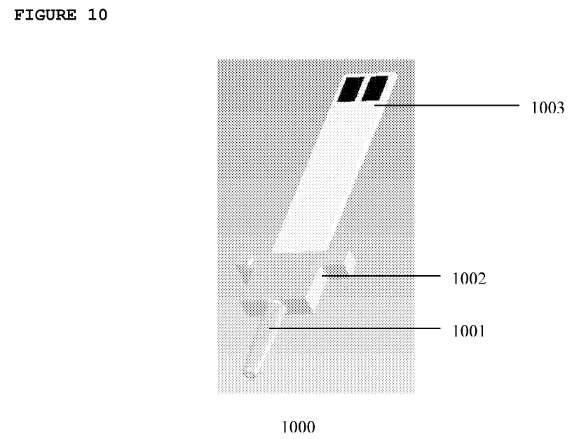

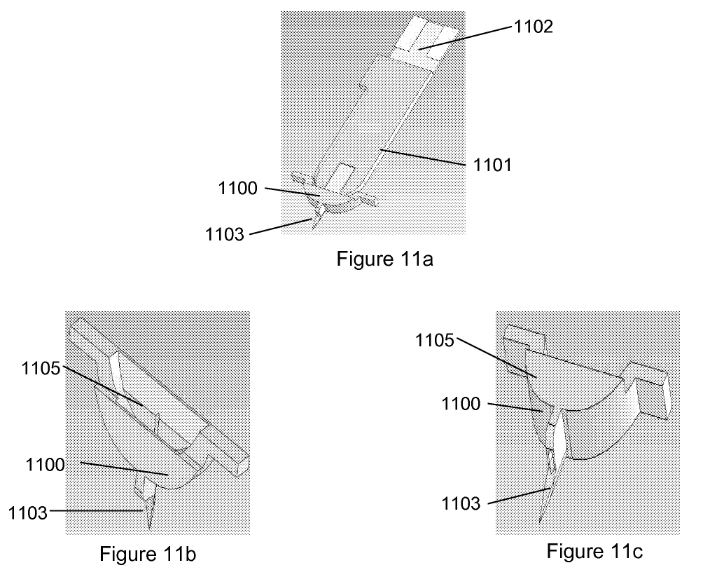

In another embodiment, shown in FIGS. 10 and 11a-11c, the testing strip is incorporated into a housing that includes a sharp projection capable of functioning as a lancet. FIG. 10 illustrates another embodiment of a test strip holder, integrated with a blood transfer tube, that can be used with the automated blood parameter testing apparatus of the present invention. Device 1000 comprises a single use transfer tube 1001, which is used to access fluid from a pierced portion of a patient's skin and transfer it to a test strip 1003, held by housing 1002, for blood glucose measurement. In FIG. 11a, the single use transfer tube of FIG. 10 is further integrated with a sharp lancing device which can be used to pierce the patient's skin to obtain blood. The device 1100 comprises a test strip holder 1101, a test strip for measuring blood glucose 1102, and an integrated lancet 1103 for piercing the patient's skin. FIGS. 11b and 11c depict a second and third view of the device 1100 integrated with a lancet 1103 where a curved receptacle 1105 in the device 1100 is used to receive a test strip.

It should be appreciate that the automated blood testing apparatus of the present invention can comprise a general purpose lancet housing within the cartridge, such that a variety of lancet devices can be used. Thus, in one embodiment, the blood testing apparatus may employ any type of lancet device depending upon patient requirement, user preference, comfort, and efficacy, among other requirements.

As shown in FIG. 8b, portable unit 800 is physically attached to a wearable cuff 802 and further comprises a display 801, compartment door 812a, and compartment 812b, wherein compartment 812b is employed to house cartridge 803. It should be appreciated that the portable unit 800 need only have some area encompassed by the housing within which a cartridge can be received and installed.

FIG. 8c illustrates cartridge 803 when properly positioned and seated into compartment 812b. In addition, portable unit 800 further comprises control buttons 804 for operator input, i.e. initiating a blood pressure reading, initiating a glucose reading, recalling prior measurements and displaying specific data.

FIGS. 8d, 8e, 8f, and 8g illustrate the operational steps of the automated blood parameter testing apparatus when in use on a patient. As mentioned above, one embodiment of the automated blood parameter testing apparatus of the present invention can comprise an inflatable arm-band or cuff. As shown in FIG. 8d, the inflatable arm-band or cuff 802 of the apparatus is fastened around the arm of the patient 810. The cuff may be fastened by any appropriate means as are well-known to those of ordinary skill in the art, including, but not limited to Velcro®.

In one embodiment, the apparatus is programmed to automatically take blood pressure and blood analyte readings at predetermined time periods. In another embodiment, the apparatus may be operated manually.

As shown in FIGS. 8e and 8f, once the apparatus 800 is fastened onto the patient 810, the compartment housing cover is opened to expose the compartment 815. A new cartridge 825 for blood sampling and measurement is inserted into the compartment 830. In one embodiment, the cartridge is pre-loaded into the apparatus prior to use on a patient. As shown in FIG. 8g, once the cartridge is loaded, the compartment cover is closed 840 and the device is initiated 835. The device can then be used to draw a blood sample, measure the level of analyte in the blood, and measure blood pressure.

In one embodiment, each lancet and test strip pair is single use. Thus, each time the apparatus is used for measuring a blood analyte, a new lancet is automatically launched for withdrawing the requisite blood sample and a new test strip is used to measure the blood analyte. In one embodiment, the test strips are pre-set into a fixed position relative to each lancet, such that, upon the lancet piercing the patient's skin, the blood sample is directed to the test strip in a fixed relation to the piercing lancet. In another embodiment, the test strips are not in a fixed relation to a specific lancet and, instead, are stepped into place as required. The test strips reside in a test strip pool and then individually moved into contact with a blood sample, as required.

After all of the lancet and test strip pairs have been used, the apparatus provides an indication that the disposable cartridge needs to be replaced. Such indication can be in any visual or auditory form, including a flashing light of any color, an alarm, or a combination thereof. If the cartridge is empty, the device will only read blood pressure and, preferably, communicates a signal to replace the cartridge, including a visual alarm, an auditory alarm, or shutting down the device.

Because the lancet and test strip pairs are placed at some distance from each other, a different area of the skin will be pierced for each measurement. It is preferred that the lancet remain in the skin for as short a time as possible

In one embodiment, each lancet is pre-treated or coated with an anticoagulant medication, to ease blood sampling. In another embodiment, each lancet is pre-treated or coated with a pain killer or anesthetic such as lidocaine, to make the test apparatus more comfortable for patients.

One mechanism for drawing a blood sample from the patient has already been described with respect to FIG. 7. Briefly, when the device is initiated to take a reading, either manually or automatically, the pressure cuff is inflated, facilitating blood flow to the skin surface. Pressurizing the cuff causes the underlying skin to protrude slightly through the access hole provided for measurement. This protrusion changes the geometry favorably and aids in obtaining the sample. In the device of the present invention, since the measuring element or test strip and lancet are arranged in a “V” shape relative to one another, the blood sample is channeled toward the test strip and thus, no separate mechanism is required to transport the withdrawn blood sample to the test strip.

In another embodiment, the lancet includes a vibrating mechanism, increasing access to the skin. The vibration mechanism of the lancet can be likened to a mosquito bite, wherein the mosquito vibrates its suction tube to penetrate the skin and find a blood source quickly and efficiently.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

What is claimed is:

1. An automated blood testing device comprising:
a sampling and measurement unit for obtaining a blood sample and measuring blood analytes in said sample, wherein the sampling and measurement unit further comprises a plurality of lancet and blood analyte measuring element pairs; and
a control unit for controlling the periodic sampling of blood and measurement of blood analytes.

2. The automated blood testing device of claim 1 wherein the control unit is programmable to initiate blood sampling for measurement of blood analytes at pre-determined time intervals.

3. The automated blood testing device of claim 1 wherein the control unit is programmable to initiate blood sampling for measurement of blood analytes based upon a pre-defined event.

4. The automated device of claim 1 wherein the lancet in each pair withdraws blood from a different location for each sample.

5. The automated device of claim 1 wherein the lancet vibrates to withdraw a blood sample.

6. The automated device of claim 1 wherein the lancet is a single-use lancet.

7. The automated device of claim 1 wherein lancet is disposable.

8. The automated device of claim 1 wherein lancet is contained in a disposable cartridge or cassette.

9. The automated device of claim 1 wherein the blood analyte measurement element is a glucose oxidase test strip.

10. The automated device of claim 1 wherein the lancet and blood analyte measurement element in each pair is arranged in a “V” configuration.

11. The automated device of claim 1 wherein each lancet is coated with an anticoagulant.

12. The automated device of claim 1 wherein each lancet is coated with an anesthetic.

13. The automated device of claim 1 wherein each lancet is provided with a flexible cover that deforms to expose the lancet tip when the lancet is actuated for sampling.

14. The automated device of claim 1 wherein the device is wearable.

15. The automated device of claim 1 wherein the device further comprises an inflatable cuff.

16. The automated device of claim 15 wherein the inflatable cuff is used for obtaining a blood sample via applying pressure.

17. The automated device of claim 15 wherein the inflatable cuff comprises a plurality of cavities, wherein each cavity can be individually inflated.

18. The automated device of claim 15 wherein the inflatable cuff is used for non-invasive measurement of blood pressure.

19. The automated device of claim 15 wherein the inflatable cuff comprises a warming pad.

20. An automated device for obtaining a blood sample and measuring blood analytes and blood parameters in said sample, comprising:
a plurality of lancets in physical proximity for drawing a blood sample;
a plurality of blood analyte measuring elements, each in physical proximity to at least one of said lancets; and
at least one processor for calculating numerical value of the blood analyte measured by said blood analyte measuring element.

21. The automated device of claim 20 wherein the lancet, the blood analyte measuring element and the processor are integrated into a single unit.

22. The automated device of claim 21 wherein said unit is replaceable.

23. The automated device of claim 22 wherein said unit is disposable.

24. The automated device of claim 21 wherein said unit is capable of being connected to a physiological parameter monitoring device.

25. The automated device of claim 20 wherein said plurality of lancets and said plurality of blood analyte measuring elements are contained in a cassette and wherein said processor is contained a housing capable of detachably receiving said cassette.

26. The automated device of claim 25 wherein said cassette is replaceable.

27. The automated device of claim 26 wherein said cassette is disposable.

28. The automated device of claim 25 wherein said cassette is assigned a unique code.

29. The automated device of claim 28 wherein said unique code is stored either in mechanical or electrical form.

30. The automated device of claim 28 wherein said unique code is used by the device to determine if the cassette is authentic.

31. The automated device of claim 25 wherein said cassette comprises calibration information.

32. The automated device of claim 31 wherein said calibration information is communicated to the device to enable the at least one processor to accurately calculate the numerical value of the blood analyte measured by said blood analyte measuring element.
